(19) United States
(12) Patent Application Publication
(10) Pub. No.: US 2010/0063116 A1
(43) Pub. Date: Mar. 11, 2010

(54) USE OF PRAMIPEXOLE OR A SALT THEREOF FOR THE TREATMENT OF PARKINSON'S DISEASE

(75) Inventors: Jeffrey G. Huth, Hopewell Junction, NY (US); Elizabeth Keating, Newtown, CT (US)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERNATIONAL GMBH, Ingelheim (DE)

(21) Appl. No.: 12/447,331

(22) PCT Filed: Oct. 29, 2007
(86) PCT No.: PCT/EP07/61584

§ 371 Date: May 15, 2009

Related U.S. Application Data
(60) Provisional application No. 60/863,451, filed on Oct. 30, 2006.
(60) Provisional application No. 60/865,535, filed on Nov. 13, 2006.

(51) Int. Cl. A61K 31/428; A61P 25/28

(52) U.S. Cl. 514/367

(57) ABSTRACT

The present invention refers to the use of pramipexole, pramipexole hydrochloride, pramipexole dihydrochloride or pramipexole dihydrochloride monohydrate for the manufacture of a medicament for the treatment of early Parkinson's disease by a maintenance, twice daily application.

The present invention refers to the use of a medicament for the treatment of Parkinson's disease with the active ingredient selected from the group of pramipexole, pramipexole hydrochloride, pramipexole dihydrochloride or pramipexole dihydrochloride monohydrate or any other pharmaceutically acceptable salt form of pramipexole for the treatment of early Parkinson's disease.

Due to the changes in ageing statistics the number of patients suffering from mental diseases due to loss of neurons is increasing, in particular in the Western world. Many of such patients smoothly lose self-control and the ability to care for themselves. Sooner or later they are dependant on part or full care every day. Among the more known diseases is Parkinson's Disease (PD). During the progression of the disease neurons (nerve cells) in the substantia nigra degenerate, or die off. Accordingly, Parkinson's disease is a neurodegenerative disorder. Neurodegenerative means the degeneration, or death, of cerebral neurons. Parkinson's disease usually affects older people. The average age at diagnosis is about 60.

The earliest symptoms of Parkinson's disease and thus the symptoms of early Parkinson's disease are tremor, slowed movements (bradykinesia), and stiffness or rigidity/akinesia. At the beginning of the disease, the symptoms often develop on one side of the body, and progress over time to involve both sides. The tremor of Parkinson's disease is a rest tremor—the shaking occurs when the patient is not trying to use the limb, and diminishes when the limb is in use. Bradykinesia and stiffness, along with loss of some balance reflexes, can combine to cause postural instability, and increase the likelihood of falling down.

Other symptoms of Parkinson's disease include: orthostatic hypotension, or loss of blood pressure upon standing, which can cause dizziness and fainting, postural instability, difficulty in swallowing and chewing, difficulties in speaking, urinary problems, constipation, painful foot cramps, micrographia, or reduced size of handwriting, reduced voice volume, reduced facial expression, excessive sweating, constipation, decreased ability to smell, male impotence, drooling, sleep disturbance, depression, anxiety, panic attacks, late-stage dementia.

To diagnose Parkinson's disease neurological examination is necessary, including testing movements, coordination, reflexes, and other aspects of function. Unilateral (one-sided) tremor, slowed movements, and muscle stiffness are generally enough to confirm the diagnosis, with this symptoms most often related to early Parkinson's disease. Two of the three are usually considered definitive. A systematic approach to define the stage of the Parkinson's disease are the modified Hoehn and Yahr scale or the Unified Parkinson Disease Rating Scale (UPDRS).

The modified Hoehn and Yahr system comprises stages ranging from 1 to 5. 1 means least severe and stage 5 means most severe stage. Stage 1 symptoms are signs and symptoms preferably on one side only, mild symptoms, symptoms inconvenient but not disabling, usually present with tremor of one limb, friends have noticed changes in posture, locomotion and facial expression. Stage 2 symptoms are bilateral, minimal disability, posture and gait affected. In the present context patients having a score or 1 or 2 according to the Hoehn and Yahr system are supposed to be in early stage.

Stage 3 symptoms are significant slowing of body movements, early impairment of equilibrium on walking or standing, generalized dysfunction that is moderately severe. Stage 4 symptoms are severe symptoms, can still walk to a limited extent, rigidity and bradykinesia, no longer able to live alone, tremor may be less than earlier stages. Stage 5 symptoms are cachectic stage, invalidism complete, cannot stand or walk, requires constant nursing care.

The Unified Parkinson Disease Rating Scale is a rating tool to follow the longitudinal course of Parkinson's Disease. It is made up of the following sections: I mentation, behavior, and mood, II activities of daily living and III motor. Part II of the UPDRS contains 13 questions relating to activities of daily living (ADL), which are scored from 0 (normal) to 4 (maximal severity) for a maximum (worst) score of 52. Part III of the UPDRS contains 27 questions (for 14 items) and is scored as described for part II. It is designed to assess the severity of the cardinal motor findings in patients with Parkinson's disease (e.g., tremor, rigidity, bradykinesia, postural instability, etc.), scored for different body regions, and has a maximum (worst) score of 108.

In the context of the present invention the treatment of early Parkinson's disease, even not-yet symptomatic Parkinson's disease is of most interest.

The current invention includes the manufacture of a medicament concerning the prophylactic treatment or the deceleration of the progression of cerebral neuronal degradation associated with early Parkinson's disease and/or for to stop further cerebral neuronal degradation associated with early Parkinson's disease and/or for to ameliorate the mental estate of an individual suffering form cerebral neuroprotection. It is also of interest to treat patients suffering from Parkinson's disease, while no typical symptoms have yet manifested (non-symptomatic treatment) or manifested shortly so that the disease still is in an early state. In the context of the present invention it is understood that typical symptoms have manifested if a physician can diagnose the disease on basis of physical and/or behavioral symptoms with a likelihood as well used in the art. The terms “typical symptoms” or “diagnosis of the disease” do not include molecular biological signs or molecular biological diagnostic methods and the like on basis of which it can be concluded that a person is predisposed to suffer from such a disease or of which it can be concluded that the disease will break out (manifest) in the future or it can be concluded that the disease might have broken out but is in a symptom-free pre-state.

A medicament with Pramipexole dihydrochloride, preferably pramipexole dihydrochloride monohydrate is known in the US under the tradename MIRAPEX® and in Germany under the tradename Sifrol®. The drug is available in form of tablets that contain pramipexole, a dopamine agonist indicated for the treatment of the signs and symptoms of idiopathic Parkinson's disease. The chemical name of pramipexole dihydrochloride is (S)-2-amino-4,5,6,7-tetrahydro-6-(propylamino)benzothiazole dihydrochloride monohydrate. Its empirical formula is C10 H17 N3 S.2HC1.H2O, and its molecular weight is 302.27. The structural formula of the free base is:

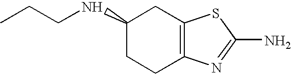

Pramipexole dihydrochloride is a white to off-white powder substance. Melting occurs in the range of 296° C. to 301° C., with decomposition. Pramipexole dihydrochloride is more than 20% soluble in water, about 8% in methanol, about 0.5% in ethanol, and practically insoluble in dichloromethane. In the following the drug substance will be called pramipexole. Pramipexole can mean the free base as well as the corresponding salts thereof as mentioned before.

The available tablets are for oral administration and so-called immediate release formulations, which means that they liberate the drug substance without significant delay once swallowed, reaching peak concentrations in approximately 2 hours. The tablets contain 0.125 mg, 0.25 mg, 0.5 mg, 1.0 mg, or 1.5 mg of pramipexole dihydrochloride monohydrate. Inactive ingredients consist of mannitol, corn starch, colloidal silicon dioxide, povidone, and magnesium stearate.

Pramipexole is a nonergot dopamine agonist with high relative in vitro specificity and full intrinsic activity at the D2 subfamily of dopamine receptors, binding with higher affinity to D3 than to D2 or D4 receptor subtypes. The relevance of D3 receptor binding in Parkinson's disease is unknown.

The precise mechanism of action of pramipexole as a treatment for Parkinson's disease is unknown, although it is believed to be related to its ability to stimulate dopamine receptors in the striatum. This conclusion is supported by electrophysiologic studies in animals that have demonstrated that pramipexole influences striatal neuronal firing rates via activation of dopamine receptors in the striatum and the substantia nigra, the site of neurons that send projections to the striatum.

Pramipexole is rapidly absorbed. The absolute bioavailability of pramipexole is greater than 90%, indicating that it is well absorbed and undergoes little presystemic metabolism. Food does not affect the extent of pramipexole absorption, although the time of maximum plasma concentration (Tmax) is increased by about 1 hour when the drug is taken with a meal.

Pramipexole is extensively distributed, having a volume of distribution of about 500 L (coefficient of variation [CV]=20%). It is about 15% bound to plasma proteins. Pramipexole distributes into red blood cells as indicated by an erythrocyte-to-plasma ratio of approximately 2.

Pramipexole displays linear pharmacokinetics over the clinical dosage range. Its terminal half-life is about 8 hours in young healthy volunteers and about 12 hours in elderly volunteers. Steady-state concentrations are achieved within 2 days of dosing.

Urinary excretion is the major route of pramipexole elimination, with 90% of a pramipexole dose recovered in urine, almost all as unchanged drug. Nonrenal routes may contribute to a small extent to pramipexole elimination, although no metabolites have been identified in plasma or urine. The renal clearance of pramipexole is approximately 400 mL/min (CV=25%), approximately three times higher than the glomerular filtration rate. Thus, pramipexole is secreted by the renal tubules, probably by the organic cation transport system.

Because therapy with pramipexole is initiated at a subtherapeutic dosage and gradually titrated upward according to clinical tolerability to obtain the optimum therapeutic effect, adjustment of the initial dose based on gender, weight, or age is not necessary. However, renal insufficiency, which can cause a large decrease in the ability to eliminate pramipexole, may necessitate dosage adjustment

The pharmacokinetics of pramipexole were comparable between early and advanced Parkinson's disease patients.

The clearance of pramipexole was about 75% lower in patients with severe renal impairment (creatinine clearance approximately 20 mL/min) and about 60% lower in patients with moderate impairment (creatinine clearance approximately 40 mL/min) compared with healthy volunteers. A lower starting and maintenance dose is recommended in these patients. In patients with varying degrees of renal impairment, pramipexole clearance correlates well with creatinine clearance. Therefore, creatinine clearance can be used as a predictor of the extent of decrease in pramipexole clearance. Pramipexole clearance is extremely low in dialysis patients, as a negligible amount of pramipexole is removed by dialysis. Caution should be exercised when administering pramipexole to patients with renal disease.

The effectiveness of the immediate release pramipexole dihydrochloride tablets as described above in the treatment of Parkinson's disease was evaluated in a multinational drug development program consisting of seven randomized, controlled trials. Three were conducted in patients with early Parkinson's disease who were not receiving concomitant levodopa, and four were conducted in patients with advanced Parkinson's disease who were receiving concomitant levodopa. Among these seven studies, three studies provide the most persuasive evidence of Pramipexole's effectiveness in the management of patients with Parkinson's disease who were and were not receiving concomitant levodopa. Two of these three trials enrolled patients with early Parkinson's disease (not receiving levodopa), and one enrolled patients with advanced Parkinson's disease who were receiving maximally tolerated doses of levodopa. In all studies, the Unified Parkinson's Disease Rating Scale (UPDRS), or one or more of its subparts, served as the primary outcome assessment measure.

Patients with Early Parkinson's Disease (N=599) in the two studies of early Parkinson's disease had a mean disease duration of 2 years, limited or no prior exposure to levodopa (generally none in the preceding 6 months), and were not experiencing the “on-off” phenomenon and dyskinesia characteristic of later stages of the disease. One of the two early Parkinson's disease studies (N=335) was a double-blind, placebo controlled, parallel trial consisting of a 7-week dose-escalation period and a 6-month maintenance period. Patients could be on selegiline, anticholinergics, or both, but could not be on levodopa products or amantadine. Patients were randomized to Pramipexole or placebo. Patients treated with Pramipexole had a starting daily dose of 0.375 mg and were titrated to a maximally tolerated dose, but no higher than 4.5 mg/day in three divided doses. At the end of the 6-month maintenance period, the mean improvement from baseline on the UPDRS part II (ADL) total score was 1.9 in the group receiving Pramipexole and −0.4 in the placebo group, a difference that was statistically significant. The mean improvement from baseline on the UPDRS part III total score was 5.0 in the group receiving Pramipexole and −0.8 in the placebo group, a difference that was also statistically significant. A statistically significant difference between groups in favor of Pramipexole was seen beginning at week 2 of the UPDRS part II (maximum dose 0.75 mg/day) and at week 3 of the UPDRS part III (maximum dose 1.5 mg/day).

The second early Parkinson's disease study (N=264) was a double-blind, placebo-controlled, parallel trial consisting of a 6-week dose-escalation period and a 4-week maintenance period. Patients could be on selegiline, anticholinergics, amantadine, or any combination of these, but could not be on levodopa products. Patients were randomized to 1 of 4 fixed doses of Pramipexole (1.5 mg, 3.0 mg, 4.5 mg, or 6.0 mg per day) or placebo. At the end of the 4-week maintenance period, the mean improvement from baseline on the UPDRS part II total score was 1.8 in the patients treated with Pramipexole, regardless of assigned dose group, and 0.3 in placebo-treated patients. The mean improvement from baseline on the UPDRS part III total score was 4.2 in patients treated with Pramipexole and 0.6 in placebo-treated patients. No dose-response relationship was demonstrated. The between-treatment differences on both parts of the UPDRS were statistically significant in favor of Pramipexole for all doses. No differences in effectiveness based on age or gender were detected. There were too few non-Caucasian patients to evaluate the effect of race. Patients receiving selegiline or anticholinergics had responses similar to patients not receiving these drugs.

As many drugs also Pramipexole is not free from side effects. So patients may falling asleep while engaged in activities of daily living, somnolence (at doses above 1.5 mg/day), drowsiness, orthostatic hypotension, especially during dose escalation, hallucinations, a single case of rhabdomyolysis occurred in a 49-year-old male with advanced Parkinson's disease treated with Pramipexole (pramipexole dihydrochloride) tablets.

Since pramipexole is eliminated through the kidneys, caution should be exercised when prescribing Pramipexole to patients with renal insufficiency

Pramipexole may potentiate the dopaminergic side effects of levodopa and may cause or exacerbate preexisting dyskinesia. Decreasing the dose of levodopa may ameliorate this side effect.

Cases of pathological gambling, hypersexuality, and compulsive eating (including binge eating) have been reported in patients treated with dopamine agonist therapy, including pramipexole therapy. As described in the literature, such behaviors are generally reversible upon dose reduction or treatment discontinuation.

The Pramipexole immediate release tablets are to be taken after instruction.

Pramipexole tablets may be subject to drug interactions as outlined in the prior art.

The immediate release Pramipexole tablets mentioned before have been used and currently are recommended to be taken as follows:

In all clinical studies, dosage was initiated at a subtherapeutic level to avoid intolerable adverse effects and orthostatic hypotension. The tablets should be titrated gradually in all patients. The dosage should be increased to achieve a maximum therapeutic effect, balanced against the principal side effects of dyskinesia, hallucinations, somnolence, and dry mouth. Dosages should be increased gradually from a starting dose of 0.375 mg/day given in three divided doses and should not be increased more frequently than every 5 to 7 days. A suggested ascending dosage schedule that was used in clinical studies is shown in the following table:

Ascending Dosage Schedule of pramipexole

| Week | Dosage (mg) | Total Daily Dose (mg) |
|---|---|---|
| 1 | 0.125 | 0.375 |
| 2 | 0.25 | 0.75 |
| 3 | 0.5 | 1.50 |
| 4 | 0.75 | 2.25 |
| 5 | 1.0 | 3.0 |
| 6 | 1.25 | 3.75 |
| 7 | 1.5 | 4.50 |

### Maintenance Treatment

The daily dosage shall administered in equally divided doses three times per day with or without concomitant levodopa (approximately 800 mg/day). In a fixed-dose study in early Parkinson's disease patients, doses of 3 mg, 4.5 mg, and 6 mg per day of Pramipexole were not shown to provide any significant benefit beyond that achieved at a daily dose of 1.5 mg/day.

Pramipexole (pramipexole dihydrochloride) tablets are available as follows:

0.125 mg, 0.25 mg, 0.5 mg, 1 mg, 1.5 mg.

All the aforementioned data and clinical trials suggest a three times a day application of Pramipexole.

However, the current invention recommends to change the application scheme for a maintenance treatment of patients with early Parkinson's disease from thrice daily to twice daily, while providing a non-inferior efficacy than the thrice a day dosage in the early Parkinson's disease patient group. There is no hint in the prior art which suggest such a new twice daily application scheme as described and also the fact that health authorities like the FDA or EMEA have authorized the thrice daily application will prevent the ordinary skilled person of the art from turning to such a new application scheme.

It will be appreciated, that when a patient starts a Pramipexol therapy for the first time, he will initially start with a subtherapeutic dosage and gradually will be titrated upward according to clinical tolerability to obtain the optimum therapeutic effect as it was necessary for the thrice daily application scheme before. The adjustment of the initial dose based on gender, weight, or age is not necessary.

The patient should profit by the twice daily in several aspects among which is that the new dosing scheme will have a more or less unchanged efficacy profile while the compliance will increase as well as a more favourable side effect profile may be observed. In a clinical trial the effectiveness of this new application scheme is investigated.

Accordingly, the present inventions refers to the use of a medicament for the treatment of Parkinson's disease with the active ingredient selected from the group of pramipexole, pramipexole hydrochloride, pramipexole dihydrochloride or pramipexole dihydrochloride monohydrate or any other pharmaceutically acceptable salt form of pramipexole for a medicament for the treatment of early Parkinson's disease, characterised in that the medicament is an immediate release formulation for a maintenance, twice daily application.

As active ingredient is pramipexole hydrochloride is preferred, pramipexole dihydrochloride is more preferred and pramipexole dihydrochloride monohydrate is even more preferred.

In the context of the present invention the term maintenance refers to a continuous application over a period of at least 12 weeks with the same every day application scheme and the same every day dosage. As outlined above the therapy starts with dosage titration.

The new application scheme is tested in a randomized, double-blind study with patients being treated with pramipexole 0.5 mg, respectively 0.75 mg (pramipexole dihydrochloride monohydrate) twice daily over a period of 12 weeks in early Parkinson's disease patients. Simultaneously the scheme is controlled via patients treated with placebo and some patients being treated with 0.5 mg thrice daily.

The efficacy of pramipexole given two times daily is compared to placebo as well as to a thrice daily application. Additionally, the effects of pramipexole on mood, cognition, fatigue, impulse control, daytime sleepiness, and nighttime sleep are compared the same way. Finally, the tolerability profile is observed.

The patient treated are women and men over 30 years of age with idiopathic Parkinson's disease of less than 7 years duration, characterized by 2 of the following 3 cardinal signs (signs need to be asymmetric): resting tremor, bradykinesia and rigidity. The patients must have Modified Hoehn and Yahr stage <3 and be able to safely tolerate placebo for up to 12 weeks after Baseline. Women of child-bearing potential must have a negative pregnancy test at Screening Visit and use adequate contraceptive methods throughout the study.

Patients may concomitantly be treated with one or more of the following medications: MAO-B inhibitors, anticholinergics, amantadine.

The dosage scheme for the patients treated with 0.5 mg twice daily (bid) or 0.75 mg twice daily (bid) or 0.5 mg thrice daily (tid) is:

| Week | 0.5 bid | 0.75 bid | 0.5 mg tid |
|---|---|---|---|
| 1 | 0.125 bid | 0.125 bid | 0.125 tid |
| 2 | 0.25 bid | 0.25 bid | 0.25 tid |
| 3 | 0.5 bid | 0.5 bid | 0.5 tid |
| 4-12 | 0.5 bid | 0.75 bid | 0.5 tid |

After the initial 4 week titration period, each dosage group maintains the specified dosage for an additional 8 weeks, to complete the 12 week double-blind period.

The efficacy of the new application scheme can be evaluated in terms of any of the following:
change in total UPDRS score from Baseline to Week 12
changes from Baseline to 12 weeks in each of the following variables:
   UPDRS component scores (mental, motor, ADL)
   Modified Hoehn and Yahr Stage
   Modified Schwab and England ADL score
   Epworth Sleepiness Scale
   Parkinson Fatigue Scale (PFS-16)
   Parkinson's Disease Sleep Scale (PDSS)
   Montreal Cognitive Assessment (MoCA)
   Beck Depression Inventory II (BDI II)
   Parkinson's Disease Questionnaire (PDQ-39)
   The Apathy Scale
   The Snaith-Hamilton Pleasure Scale (SHAPS)
   The Modified Minnesota Impulsive Disorders Interview (MMIDI)

Concerning the Unified Parkinson's Disease Rating Scale (UPDRS) only the first three parts are used for patients with early PD. For Parts I-III combined, the total scores range from 0 to 176.

The Modified Hoehn and Yahr has been explained above

The Modified Schwab and England Activities of Daily Living Scale reflects the speed, ease, and independence with which an individual performs daily activities or personal chores, such as eating, toileting, and dressing. This scale uses a rating scale from 0% to 100%, with 100% representing complete independence in performing daily activities and 0% representing a vegetative, bedridden state.

The Epworth Sleepiness Scale (ESS), used extensively in PD related studies, is a self-administered questionnaire collecting information on the propensity to fall asleep in eight different situations encountered commonly in daily life. Each situation is rated from 0 (no chance of dozing) to 3 (high chance of dozing), and the total score ranges from 0 to 24.

The Parkinson Fatigue Scale (PFS-16) was designed to assess the physical aspects of fatigue and their impact on the daily function of the patient with PD. It deliberately excludes emotional and cognitive features that may occur independently in parkinsonism. The scale is a 16-item self-report instrument with higher scores indicating greater fatigue.

The Parkinson's Disease Sleep Scale (PDSS) is a self-administered visual analogue scale addressing 15 commonly reported aspects of nocturnal sleep difficulties in PD patients. Study subjects mark severity for each item by placing a cross mark on a 10 cm line labeled from worst to best state. The maximum total score is 150 indicating the subject is free of all symptoms.

The Montreal Cognitive Assessment (MoCA): In early Parkinson's disease, when cognitive deficits occur, they are subtle and mild and the patients usually perform in the normal range on the widely used Mini Mental State Examination. The Montreal Cognitive Assessment (MoCA) is a rapid screening instrument like the MMSE but was developed to be more sensitive to patients presenting with mild cognitive complaints. It assesses short term and working memory, visuospatial abilities, executive function, attention, concentration, language and orientation. The total score ranges from 0 to 30.

The Beck Depression Inventory II (BDI II) is a screening and diagnostic scale covering several depressive dimensions (somatization, cognition, motivation). Each of the 21 items on this self-rating scale is scored from 0 (absent) to 3 (severe); the total score ranges from 0 to 63.

The Parkinson's Disease Questionnaire (PDQ-39) is disease-specific instrument designed to measure aspects of health that are relevant to patients with PD, and that may not be included in general health status questionnaires. This self-administered questionnaire contains 39 items evaluating eight areas (mobility, activities of daily living, emotional wellbeing, stigma, social support, cognition, communication, and bodily discomfort). Higher scores are consistently associated with the more severe symptoms of PD, while lower scores indicate a better perceived health status.

The Apathy Scale (AS) to be used in this study has been found to be reliable and valid in the diagnosis of apathy in PD patients, and is an abridged version of the Apathy Evaluation Scale developed by Marin (R06-1342). The 14 items are read to the patient by the examiner. The possible answers are “not at all,” “slightly,” “some,” and “a lot.” Total scores range from 0 to 42 with higher scores indicating more severe apathy.

The Snaith-Hamilton Pleasure Scale (SHAPS) is a self-administered scale designed to measure the individual's ability to experience pleasure in the last few days. The 14 items are answered as “disagree” or “strongly disagree” (scored 1) or “agree” or “strongly agree” (scored 0). Total scores range from 0 to 14 with higher scores indicating more anhedonia.

The Minnesota Impulsive Disorders Interview is a semi-structured clinical interview with modules that screen for pathological gambling, trichotillomania, kleptomania, pyromania, intermittent explosive disorder, compulsive buying, and compulsive sexual behavior.

Given the objective of the invention, that a twice daily application of pramipexole is similar effective as a thrice daily application in early Parkinson's disease patients, any means to measure the efficacy and any parameter used for the new application scheme is comparable to the means and parameters known from the thrice daily application.

1. A method for treating early Parkinson's disease in a patient, comprising administering to said patient an effective amount of an immediate release formulation containing an active ingredient selected from the group consisting of pramipexole, pramipexole hydrochloride, pramipexole dihydrochloride and pramipexole dihydrochloride monohydrate, or any other pharmaceutically acceptable salt form of pramipexole, wherein the formulation is administered to said patient twice daily as a maintenance treatment.

2. The method according to claim 1, wherein the total every day dosage is between 0.2 to 5.0 mg.

3. The method according to claim 1, wherein the total every day dosage is between 0.25 to 2.5 mg.

4. The method according to claim 1, wherein the total every day dosage is between 0.5 to 1.5 mg.

5. The method according to claim 1, wherein the total every day dosage is between 0.5 and 0.8 mg.

6. The method according to claim 1, wherein the total every day dosage is applied in two equal amounts.

7. The method according to claim 1, wherein the period in-between the two takings is between 7 to 17 hours.

8. The method according to claim 1, wherein the period in-between the two takings is between 8 to 16 hours.

9. The method according to claim 1, wherein the period in-between the two takings is between 9 to 15 hours.

10. The method according to claim 1, wherein the period in-between the two takings is between 10 to 14 hours.

11. The method according to claim 1, wherein the period in-between the two takings is between 11 to 13 hours.

12. The method according to claim 1, wherein the period in-between the two takings is 12 hours.

13-18. (canceled)

19. The method according to claim 1, wherein the immediate release formulation is a tablet which comprises 0.125 mg, 0.25 mg, 0.5 mg, 1.0 mg, or 1.5 mg of pramipexole dihydrochloride monohydrate and the inactive ingredients mannitol, corn starch, colloidal silicon dioxide, povidone, and magnesium stearate.

20. A kit comprising an immediate release formulation containing an active ingredient selected from the group consisting of pramipexole, pramipexole hydrochloride, pramipexole dihydrochloride and pramipexole dihydrochloride monohydrate, or any other pharmaceutically acceptable salt form of pramipexole, and an instruction to administer the immediate release formulation twice daily.

21. The kit according to claim 20, wherein the instruction is such that the total every day dosage is between 0.2 to 5.0 mg.

22. The kit according to claim 20, wherein the instruction is such that the total every day dosage is between 0.25 to 2.5 mg.

23. The kit according to claim 20, wherein the instruction is such that the total every day dosage is between 0.5 to 1.5 mg.

24. The kit according to claim 20, wherein the instruction is such that the total every day dosage is between 0.5 and 0.8 mg.

25. The kit according to any of claim, wherein the instruction is such that the total every day dosage is applied in two equal amounts.

26-37. (canceled)

38. The kit according to claim 20, wherein the immediate release formulation is a tablet which comprises 0.125 mg, 0.25 mg, 0.5 mg, 1.0 mg, or 1.5 mg of pramipexole dihydrochloride monohydrate and the inactive ingredients mannitol, corn starch, colloidal silicon dioxide, povidone, and magnesium stearate.

39-40. (canceled)
